(19) United States
(12) Patent Application Publication
(10) Pub. No.: US 2015/0047636 A1
(43) Pub. Date: Feb. 19, 2015

(54) Inhalation Device

(71) Applicant: Sanofi SA, Vemier (CH)

(72) Inventor: Stefan Mayer, Freiburg Im Breisgau (DE)

(21) Appl. No.: 14/372,011

(22) PCT Filed: Jan. 20, 2013
(86) PCT No.: PCT/EP2013/051128

§ 371 Date: Jul. 12, 2014

(30) Foreign Application Priority Data
Jan. 23, 2012 (EP) 21252136.3

(51) Int. Cl. A61M 15/00

(52) U.S. Cl. CPC A61M 15/0081; A61M 15/0025; A61M 15/0073
USPC 128/203.14

(57) ABSTRACT

The present disclosure relates to an inhalation device for delivering at least one dose of a medicament, comprising a lock-out mechanism to prevent an operation of the inhalation device when a given number of doses has been delivered.

### CROSS REFERENCE TO RELATED APPLICATIONS

The present application is a U.S. National Phase Application pursuant to 35 U.S.C. §371 of International Application No. PCT/EP2013/051128 filed Jan. 22, 2013, which claims priority to European Patent Application No. 12152136.3 filed Jan. 23, 2012. The entire disclosure contents of these applications are herewith incorporated by reference into the present application.

### FIELD OF INVENTION

This disclosure relates to an inhalation device.

### BACKGROUND

An inhalation device is usually activated by the user's suction airstream and is intended for the inhalation of a substance, in particular a powdery substance. An inhalation device is described in document WO 2009/065707 A1, for example.

EP 1 616 592 A1 discloses an inhaler for the administration of powdered pharmaceuticals comprising a key wherein the inhaler further comprises a locking mechanism configured to block the key after a predetermined number of metering cycles.

WO 2008/077623 A1 discloses an inhalation device comprising a counting device formed by a two-digit drum counter.

### SUMMARY

It is an object of the present disclosure to provide an improved inhalation device, e.g. an inhalation device that is more user-friendly or that is improved regarding the feedback provided to a user.

This object may be achieved by the subject of present claim 1. Advantageous embodiments and refinements are the subject-matters of the dependent claims. However, further advantageous concepts may be disclosed herein besides the ones which are claimed.

One aspect of the present disclosure relates to an inhalation device for delivering at least one dose of a medicament, comprising a lock-out mechanism to prevent an operation of the inhalation device when a given number of doses, in particular a predefined number of doses, has been delivered. The operation configured to be prevented by the lock-out mechanism may be a dose delivery operation of the inhalation device.

Preferably, the operation is prevented permanently once the given number of doses has been delivered. In particular, once the lock-out mechanism has prevented the operation, the operation may only be allowed again if the housing of the device is opened and the lock-out mechanism is released. Preferably, a medicament container needs to be changed in order to unlock the lock-out mechanism. In this case, the device may be configured to be a reusable device allowing for a container change. Alternatively, the device may be configured such that once the lock-out mechanism has locked the device, i.e. prevented the operation, it is not possible to enable the operation again at all. In this case, the device may be configured to be disposed once device has been locked.

The inhalation device may be configured to carry out multiple different operations, e.g. a dose setting operation, a dose delivery operation, an operation to amend a set dose, a container replacement operation. The lock-out mechanism is configured such that it is enabled to prevent at least one of said operations. The lock-out mechanism is configured such that it allows the dose setting operation when it prevents the at least one operation. The lock-out mechanism may be configured to prevent one or more of the other operations of the inhalation device. In particular, the lock-out mechanism may be configured to prevent the dose delivery operation. When the lock-out mechanism prevents the dose delivery operation, at least the dose setting operation can still be carried out. It may also be possible to carry out some of the other operations.

The dose setting operation may be defined as an operation which provides a defined dose of the substance. The dose setting operation may be an operation which prepares a dose of the substance for dispense. In the dose setting operation a single dose of the substance may be separated from a larger reservoir of the substance containing substance for multiple doses. The dose delivery operation may be defined as an operation wherein the previously set dose of the substance is expelled from the device.

When the user performs the operation, an audible or visible feedback may be provided to the user signaling to the user that the operation was successful. However, if the operation is prevented by the lock-out mechanism, the user may be alerted that the operation is not carried out successfully by the feedback not being provided. Accordingly, the missing feedback may be an indication to the user that a given number of doses has been delivered.

The given number of doses may, in particular, correspond to the total number of doses that can be provided by the inhalation device, in particular, the number of doses being provided by a medicament container. Accordingly, the lock-out mechanism prevents the operation when further doses can not be delivered, because the device is empty.

In one embodiment, the operation configured to be prevented by the lock-out mechanism is a dose delivery operation of the inhalation device. However, a dose setting operation may still be possible. In particular, the device may be configured such that an element, in particular an element of a drive mechanism of the device, has to be moved during the operation, in particular, the dose delivery operation. The lock-out mechanism may prevent a movement of said element and, thereby, prevent the dose delivery operation.

In one embodiment, the lock-out mechanism comprises a blocking element having a blocking position, wherein when the blocking element is in the blocking position the operation of the inhalation device is prevented.

In particular, the blocking element may prevent a movement of the element that has to be moved during the operation of the device, in particular during the dose delivery operation. In particular, the blocking element may prevent a movement of the element relative to a housing of the device. As an example, the blocking element may engage with the element, e.g. the blocking element may mechanically fix the element.

In one embodiment, the blocking element further has an unblocking position allowing the operation of the inhalation device before the given number of doses has been delivered. In the unblocking position, the blocking element may not mechanically engage with the element that is configured to move in the operation, in particular the dose delivery operation. Thereby, the dose delivery operation may be allowed.

In one embodiment, the inhalation device is configured such that the blocking element changes from its unblocking position to its blocking position when the given number of doses has been delivered. In particular, the device may be configured such that the blocking element changes from its unblocking position to its blocking position when the given number of doses has been delivered and when further a cap has been screwed onto a housing of the inhalation device. Screwing the cap onto the housing may cause an update of a dose counting mechanism. When the dose counting mechanism has been updated and, thus, has counted the given number of doses, a movement of the blocking element to its blocking position may be enabled. Accordingly, the device maybe configured such that the blocking element moves into its blocking position when the given number of doses has been delivered and the dose counting mechanism has been updated.

In particular, when the given number of doses has been delivered, a path between the unblocking position and the blocking position of the blocking element may be cleared such that the blocking element is allowed to engage with the element that is moved during the operation, in particular during dose delivery.

In one embodiment, the inhalation device may further comprise a dose counting mechanism to count a number of doses, in particular to increment or decrement a value corresponding to, e.g., the number of doses left in the device or the number of doses that have been delivered by the device.

The dose counting mechanism may provide information to a user about said number of doses. Therefore, the dose counting mechanism may increase the user-friendliness of the inhalation device.

In one embodiment, the lock-out mechanism may be configured to prevent an operation of the inhalation device when the number counted by the dose counting mechanism has a first predefined numerical value. This first predefined numerical value may correspond to the given number of doses. The lock-out mechanism may also prevent a further operation of the dose counting mechanism when the counted number has a first predefined numerical value. The operation of the dose counting mechanism prevented by the lock-out mechanism may be incrementing or decrementing the number.

In one embodiment, the dose counting mechanism may comprise a counting member. The counting member may comprise a wheel. Preferably, the counting member is movable, in particular rotatable. The counting member may have at least a first and a second state, in particular a first and a second orientation. Preferably, the counting member is configured to prevent a movement of the blocking element when the counting member is in its first state. Further, when the counting member is in its second state it may allow a movement of the blocking element. As an example, when the counting member is moved from its first state to its second state, a movement of the blocking member from the unblocking position to the blocking position may be allowed. Alternatively, the movement of the counting member from its first state to its second state may allow a movement of the blocking member which is not a movement into the blocking position. Accordingly, after the allowed movement, the blocking member may still be in an unblocking position.

The counting member may provide a path which allows a movement of the blocking element when the counting member is in its second state. The path element may comprise an opening provided in the counting member, in particular a hole through the counting member. Preferably, the path is provided in the counting member in a position different from the center of the counting member. Thereby, the position of the path alters when the counting member rotates about an axis extending through the center of the counting member.

The counting member may comprise digits. In particular, the counting member may comprise digits on its side face. The side face may be a surface facing away from a symmetry axis of the counting member.

In one embodiment, the blocking element is enabled to move to its blocking position when the path and the blocking element are aligned. In particular, the blocking element and the path may be aligned when the counting member is in its second state. Preferably, the counting mechanism is configured such that the path and the blocking element are aligned when the given number of doses has been delivered and when the counting mechanism has counted the given number. The path may allow a movement of the blocking element, in particular a movement enabling contacting the element which has to be moved during the operation to be prevented by the blocking element.

In one embodiment, the dose counting mechanism may comprise a second counting member. The second counting member may have a similar shape as the first counting member.

In particular, the second counting member may provide a wheel. Preferably, the second counting member is movable, in particular rotatable. The second counting member may have at least a first and a second state, in particular a first and a second orientation. Preferably, the second counting member is configured to prevent a movement of the blocking element when the second counting member is in its first state. Further, when the second counting member is in its second state it may allow a movement of the blocking element.

The second counting member may comprise a path which allows a movement of the blocking element when the second counting member is in its second state. The path may comprise an opening provided in the second counting member, in particular a hole through the second counting member. Preferably, the path is provided in the second counting member in a position different from the center of the counting member. Thereby, the position of the path alters when the second counting member rotates about an axis extending through the center of the second counting member.

The second counting member may comprise digits. In particular, the second counting member may comprise digits on its side face. The side face may be a surface facing away from a symmetry axis of the second counting member.

The inhalation device may be configured such that the blocking element is enabled to move to its blocking position only when all of the counting members are in their respective second state. In particular, the blocking element can only move to its blocking position when the path of each of the counting members is aligned with the blocking element.

In particular, the blocking element may gradually move to its blocking position by successive movements. A first movement may be carried out when the second counting member reaches its second state. In particular, the blocking element may then be aligned with the path of the second counting member. However, this movement may not cause an engagement of the blocking member and the element that is moved during the operation, thereby not preventing a further movement of the element. In particular, an engagement of the blocking member and the element that is moved during the operation may be prevented by the first counting member being in its first state. Accordingly, the blocking member may still be in an unblocking position.

A second movement may be carried out when the first counting member reaches its second state. In particular, the blocking element may then be aligned with the path of the first counting member. This movement may cause an engagement of the blocking member and the element that is moved during the operation, thereby preventing a further movement of the element that is moved during the operation. Accordingly, the blocking member is then in its blocking position.

In one embodiment, the dose counting mechanism may comprise an interaction member and a coupling member. The interaction member may be configured to interact with the coupling member when the interaction member is moved in a first direction.

The first direction may be a proximal direction. In this context, the term “proximal end” may refer to the end of the inhalation device or the end of a part of the device furthest away from a dispensing end of the device. Accordingly, a movement in a proximal direction is a movement towards the proximal end. Further, the term “distal end” may refer to the end of the inhalation device or a part of the inhalation device closest to the dispensing end. Accordingly, a movement in a distal direction is a movement towards the distal end.

The interaction member may be moved in the first direction after a dose delivery operation has been completed. In particular, the interaction member may be moved in the first direction when a user performs an after-dose delivery operation, e.g. screws a cap onto a housing of the device.

The coupling member may be coupled to the counting members and, thereby, transmit a movement of the interaction member to the counting members such that the counted number is incremented or decremented. Alternatively, the coupling member may be provided by a counting member.

The coupling member may comprise a toothed wheel or a Geneva wheel, for example. A movement of the interaction member in a first direction may be transferred into a movement of the coupling member, in particular a rotational movement of the coupling member. Further, a movement of the coupling member may be transferred into a movement of at least one of the counting members.

In particular, the coupling member may be coupled to at least one of the counting members directly or by a gearing. The gearing may transfer a rotation of the coupling member by a first angle into a rotation of the counting member by a second angle.

The interaction member may be enabled to engage with the coupling member during at least parts of the movement of the interaction member in the first direction. The interaction member may not engage with the coupling member during its movement in the second direction.

The movement of the interaction member in the second direction may be a movement of the interaction member in a distal direction. This movement may correspond to a dose delivery operation.

The interaction member may be enabled to interact with the coupling element to change the counted number. The interaction member may preferably be enabled to rotate the coupling element by a predefined angle.

In one embodiment, the dose counting mechanism further comprises a guide track preventing an interaction of the interaction member and the coupling element when the interaction member is moved in a second direction.

In one embodiment, the inhalation device may comprise an actuating element being displaceable from a first position to a second position. The lock-out mechanism may be enabled to prevent a displacement of the actuating element from its first position to its second position when the given number of doses has been delivered. The actuating element may comprise or be the element that has to be moved during the dose delivery operation. The actuating element may be displaceable by a user's suction airstream. The interaction member may be provided by the actuating element. Preferably, the interaction member is an integral part of the actuating element or may be fixed to the actuating element. Accordingly, the interaction member is not enabled to move axially relative to the actuating element. Therefore, if the actuating element is moved axially in the proximal or distal direction, the interaction member follows said movement.

In one embodiment, the lock-out mechanism may further comprise a biasing member being coupled to the blocking element. The biasing member may exert a biasing force on the blocking element, in particular, when the blocking member is in the unblocking position. The biasing member may comprise a spring. The biasing member may be tensed when the blocking member is in the unblocking position of the lock-out mechanism. Further, the biasing member may be allowed to relax when the blocking element moves to its blocking position.

### BRIEF DESCRIPTION OF THE DRAWINGS

In one embodiment, the biasing member may be configured to move the blocking element from its unblocking position to its blocking position when the given number of doses has been delivered. In particular, the biasing member may move the blocking element into the paths of the at least one counting member and further into engagement with the interaction member.

FIG. 1 schematically shows a sectional side view of an inhalation device,

FIG. 2 schematically shows a sectional side view of a part of an inhalation device,

FIG. 3 schematically shows parts of the inhalation device in an exploded view,

FIG. 4 schematically shows the interaction of an interaction member and a counter drive element, and

FIG. 5 schematically shows a sectional side view of a part of the inhalation device in a locked-out state.

### DETAILED DESCRIPTION

Like elements, elements of the same kind and identically acting elements may be provided with the same reference numerals in the figures.

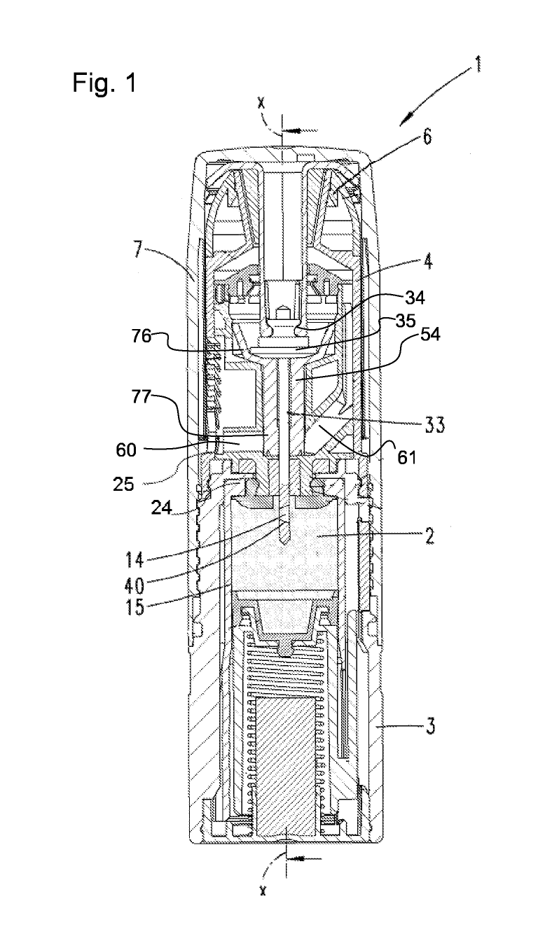

In FIG. 1, a sectional side view of an inhalation device 1 is shown.

The inhalation device 1 comprises a housing 3. The device 1 comprises an outer cylinder 4. The outer cylinder 4 is secured against axial movement with respect to the housing 3. The outer cylinder 4 is rotatable with respect to the housing 3.

The inhalation device 1 further comprises a mouthpiece 6. The inhalation device 1 comprises a cap 7. The cap 7 is used for covering the mouthpiece 6. The cap 7 may comprise a screw thread. The cap 7 may be rotatable with respect to the housing 3 for screwing the cap 7 onto the device 1 and for unscrewing the cap 7 from the device 1. The outer cylinder 4 is rotationally fixed to the cap 7. In particular, the outer cylinder 4 follows rotation of the cap 7 with respect to the housing 3. For the detailed description of the components of the inhalation device 1 and their mechanical cooperation it is referred to document WO 2009,065707 A1.

The device 1 comprises a storage chamber 15. The storage chamber 15 holds one dose, preferably a plurality of doses, of a medical substance 2. The substance 2 may be a powder.

In particular, the plurality of doses may correspond to a predefined number of doses, such that after the predefined number of doses has been delivered a lock-out mechanism may prevent a further operation of the device. The lock-out mechanism is not shown in FIG. 1, but is discussed in detail in connection with the following Figures.

A numerical value corresponding to the predefined number of doses is a starting value of a dose counting mechanism. Before the first dose delivery, the dose counting mechanism displays this predefined number as the number of available doses and with every dose delivery the number is decremented. The dose counting mechanism is not shown in FIG. 1, but the dose counting mechanism will be discussed in detail later on.

The storage chamber 15 is terminated by a chamber sealing 24. The chamber sealing 24 is formed integrally with a top wall of the storage chamber 15. The device 1 further comprises a rotary part 25. The rotary part 25 is of substantially plate-like configuration and is connected in a rotationally fixed manner to the outer cylinder 4. Accordingly, the rotary part 25 follows rotation of the cap 7 and, hence, of the outer cylinder 4 about a main longitudinal axis x of the device 1 with respect to the storage chamber 15. However, the rotary body 25 is axially fixed relative to the housing 3.

The device 1 further comprises a metering rod 33. The metering rod 33 may be connected to the cap 7 by a snap fit element 34 when the cap 7 is engaged to the housing 3. The metering rod 33 is rotatably connected to the rotary part 25 by mechanical cooperation with the rotary part 25. Accordingly, the metering rod 33 follows rotational movement of the cap 7 and, hence, of the rotary part 25 about the main longitudinal axis x when the cap 7 is mounted onto the device 1 or demounted from the device 1.

When the cap 7 is engaged to the housing 3 the metering rod 33 travels axially in the proximal direction such that the most proximal part of the metering rod 33 comprising a metering chamber 40 enters the storage chamber 15. When the cap 7 is disengaged from the housing 3 the metering rod 33 travels axially in the distal direction such that the most proximal part of the metering rod 33 exits the storage chamber 15. In this context, “distal” may refer to the end of the inhalation device closest to the mouthpiece 6. Accordingly, “proximal” may refer to the end of the inhalation device furthest away from the mouthpiece 6.

In particular, the metering rod 33 is configured for functioning as a moving metering chamber 40 for a sub-quantity 14 of the substance 2 which is to be dispensed. The metering chamber 40 is formed in that end section of the metering rod 33 which projects into the substance 2.

The inhalation device 1 further comprises a flow path comprising a flow channel 60 and an intermediate channel portion 61.

The inhalation device 1 further comprises an actuating element 54. The actuating element 54 comprises a piston comprising tongues 77 and a piston head 76. The actuating element 54 has a first and a second position. The first position is more proximal than the second position. In the first position, the tongues 77 of the actuating element 54 block the flow path between the flow channel 60 and the intermediate channel portion 61. In its second position, the actuating element 54 is positioned further distal such that the tongues 77 do not block the flow path between the flow channel 60 and the intermediate channel portion 61.

The cap 7 is disengaged from the housing 3 by unscrewing the cap 7 from the housing 3. Accordingly, the cap 7 performs concurrently an axial movement in the distal direction and a rotational movement. During disengagement of the cap 7 from the housing 3, the rotational movement of the cap 7 is transferred into a rotation of the rotary part 25 around the longitudinal axis x. The rotation of the rotary part 25 is transferred into a rotation of the actuating element 54. Further, the concurrent axial and rotational movement of the cap 7 is transferred to the metering rod 33 concurrently performing an axial movement in the distal direction and a rotational movement around the longitudinal axis x. As the cap 7 approaches the end of the threaded connection to the housing 3, the snap fit element 34 is disengaged from the metering rod 33.

During disengagement of the cap 7 from the housing 3, the actuating element 54 is not moved axially relative to the housing 3. Accordingly, the actuating element 54 is in its first position before and after disengagement of the cap 7 from the housing 3.

When the cap 7 is fully disengaged from the housing 3, the metering chamber 40 is in a first condition. The first condition of the metering chamber 40 is defined by the tongues 77 of the actuating element 54 closing the metering chamber 40 such that the metering chamber 40 is not in contact with the flow path. Accordingly, when the actuating element 54 is in its first position and the cap 7 is disengaged from the housing 3, the metering chamber 40 is in its first condition.

In the first condition of the metering chamber 40, the tongues 77 of the actuating element 54 cover over the metering chamber 40 on each side. Accordingly, in this first condition, it is not possible for the sub-quantity 14 of substance to trickle out. Rather, the substance is held captive in the metering chamber 40.

After the cap 7 has been demounted, the user may trigger an inhalation operation by subjecting the device to a suction airstream, in the simplest case by the user breathing in. Air is sucked in via the mouthpiece 6, and this, in first instance, by virtue of the piston head 76 being subjected to the action of air, results in the actuating element 54 being displaced axially in the direction of the mouthpiece 6.

By virtue of the axially displaced actuating element 54, the tongues 77 are likewise displaced axially, in order to release the metering chamber 40. The metering chamber 40 is then in a second condition. The second condition of the metering chamber 40 is defined by the actuating element 54 being in its second position. In its second condition, the metering chamber 40 lies freely in a flow path between a flow channel 60 and an intermediate channel portion 61. The metering chamber is cleared out with air being sucked from the flow channel 60.

After the inhalation operation, the cap 7 may be engaged to the housing 3. During engagement of the cap 7 to the housing 3, the cap 7 is moved axially in the proximal direction and concurrently rotated around the longitudinal axis x. The snap fit element 34 engages to the metering rod 33 at the beginning of the threaded connection. Thereby the metering rod 33 is rotated and moved into the proximal direction when the cap 7 is engaged to the housing 3.

The metering rod 33 comprises a shield 35. The axial movement of the metering rod 33 during engagement of the cap 7 to the housing 3 is mechanically transferred to the actuating element 54 by the shield 35. In particular, the actuating element 54 is pushed from its second position to its first position. If the actuating element 54 is already in its first position, it is not moved axially during engagement of the cap 7 to the housing 3.

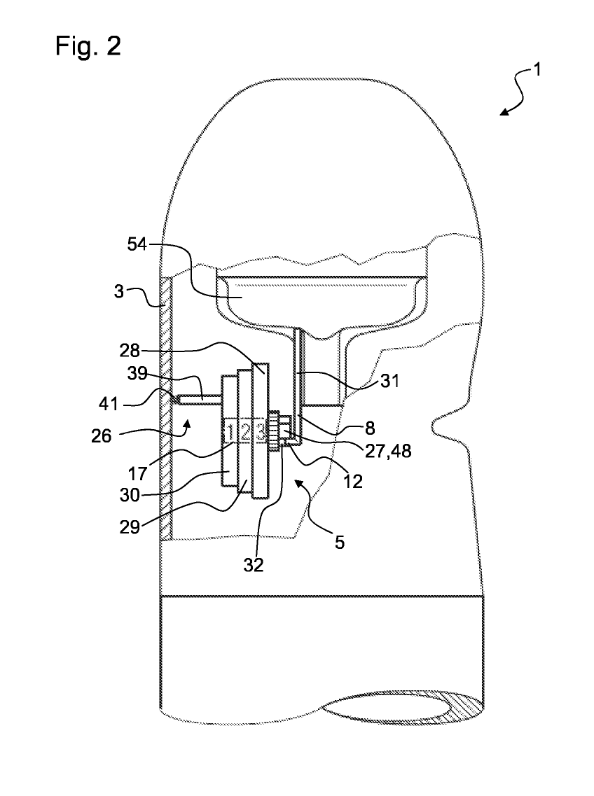

The inhalation device 1 further comprises a dose counting mechanism 5 and a lock-out mechanism 26 (see FIG. 2) which, for clarity reasons, are not depicted in FIG. 1. The dose counting mechanism 5 counts the number of doses left in the device 1. Alternatively, the counted number may correspond to another quantity, e.g. the number of doses having been delivered by the inhalation device 1.

FIG. 2 schematically shows a sectional side view of a part of the inhalation device 1. In particular, the dose counting mechanism 5 and the lock-out mechanism 26 are shown in FIG. 2. Further, FIG. 3 shows an exploded view of parts of the inhalation device 1, in particular of the counting mechanism 5 and the lock-out mechanism 26.

The dose counting mechanism 5 comprises an interaction member 8, a coupling element 27, a first counting member 28, a second counting member 29 and a third counting member 30. In particular, the first counting member 28 may comprise a first counting wheel. The second counting member 29 may comprise a second counting wheel and the third counting member 30 may comprise a third counting wheel.

The coupling element 27 is coupled to the first counting member 28. The first counting member 28 is disk-shaped and has digits on its side face. The surface normal to the side face is perpendicular to a symmetry axis of the first counting member 28. The first counting member 28 is enabled to rotate around the symmetry axis.

The interaction member 8 is provided by the actuating element 54. The interaction member 8 may be an integral part of the actuating element 54. The interaction member 8 is a deflectable element. In particular, the interaction member 8 is configured as a deflectable stick. The interaction member 8 comprises a main part 31 and a freely suspended tip 12. One end of the main part 31 is connected to the actuating element 54. At the other end of the main part 54, the freely suspended tip 12 is located, wherein the tip 12 extends at an angle relative to the main part 31. In particular, the freely suspended tip 12 forms a rectangular angle with respect to the main part 31 of the interaction member 8.

The coupling element 27 is configured to transfer a movement of the interaction member 8 into a movement of at least one of the counting members 28, 29, 30. In particular, the coupling element 27 may transfer a movement of the interaction member 8 in the proximal direction into a rotational movement of the first counting member 28. Further, the counting mechanism may be configured such that a movement of the interaction member 8 in the distal direction is not transferred into a movement of the coupling element 27 and the first counting member 28.

The coupling element 27 and the first counting member 28 are coupled by a first gearing which is not shown in FIGS. 2 and 3. Accordingly, a rotation of the coupling element 27 is transferred into a rotation of the first counting wheel 28. Preferably, the first gearing is designed such that a rotation of the coupling element 27 by an angle is transferred into a rotation of the first counting member 28 by a smaller angle. Preferably, the gearing is configured such that ten dose delivery operations correspond to a complete rotation of the first dose counting member 28 by 360°.

Moreover, the counting mechanism 5 comprises a second and a third counting member 29, 30. The second and the third counting members 29, 30 are also disk-shaped, having digits on their side faces. The second counting member 29 is coupled to the first counting member 28, e.g. by a second gearing such that a complete rotation of the first counting member 28 by 360° is transferred into a rotation of the second counting member 29 by a predefined angle. The predefined angle may be chosen such that after 100 delivered doses the second counting member is rotated by 360°.

Further, the third counting member 30 is coupled to the second counting member 29 by a third gearing such that a complete rotation of the second counting member 29 is transferred into a rotation of the third counting member 30 by a predefined angle.

In the embodiment shown in FIGS. 2-5, the coupling element 27 is a Geneva wheel 48. In an alternative embodiment which is not shown in the Figures the coupling element 27 and the first counting member 28 may be formed by a single element fulfilling both functions.

Further, the Geneva wheel 48 comprises a plurality of coupling areas. In particular, the Geneva wheel 48 comprises a plurality of receptacles 32. In particular, in the embodiment shown in FIGS. 2 to 5, the Geneva wheel 48 comprises four receptacles 32. The receptacles 32 are positioned at equally spaced circumferential positions at the Geneva wheel 48. Each receptacle 32 is sized and positioned to receive the freely suspended tip 12 of the interaction member 8 when the Geneva wheel 48 and the suspended tip 12 are in predefined specific states.

In particular, the freely suspended tip 12 of the interaction member 8 is configured to engage into a receptacle 32 of the Geneva wheel 48 and to rotate the Geneva wheel 48 by a predetermined angle, e.g. by an angle of 90°.

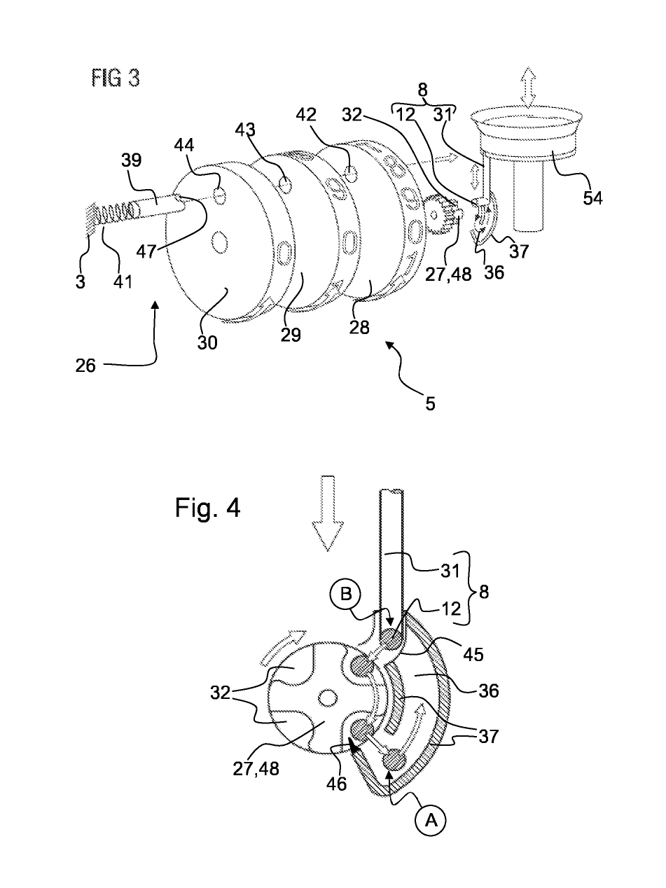

The Geneva wheel 48 is designed such that the freely suspended tip 12 engages with the Geneva wheel 48 when the interaction member 8 is moved from a distal position in a proximal direction. The distal position is the second position of the interaction member 8, marked as “B” in FIG. 4. The proximal position is the first position of the interaction member 8, marked as “A” in FIG. 4.

For this purpose, an entry guide lip 45 may be provided which initiates the engagement of the freely suspended tip 12 with the Geneva wheel 48 at the beginning of the movement of the interaction member 8 in the proximal direction.

FIG. 4 schematically shows the interaction of the interaction member 8 and the Geneva wheel 48. When the actuating element 54 is in its first position also the interaction member 8 is in a first position. The first position of the interaction member 8 is indicated as position “A” in FIG. 4. The first position of the actuating element 54 corresponds to the first condition of the metering chamber 40. This condition is present when the cap 7 has been disengaged from the housing 3 and a dose delivery has not yet been performed. Accordingly, in the first position of the actuating element 54, the tongues 77 of the actuating element 54 cover over the metering chamber 40 on each side and the sub-quantity 14 of the substance 2 is held reliably captive in the metering chamber 40.

When the actuating element 54 is in its first position, a user's suction airstream initiates a dose delivery. The user's suction airstream moves the actuating element 54 from its first position to its second position. In this position, the tongues 77 are likewise displaced axially, in order to release the metering chamber 40. The metering chamber 40 is cleared out with air being sucked from the flow channel 60. When the actuating element 54 is moved from its first position to the second position, the interaction member 8 is also moved from its first position to its second position. The second position of the interaction member 8 is indicated as position “B” in FIG. 4.

Moreover, the counting mechanism 5 comprises a guide track 36. The entry guide lip 45 is provided on the guide track 36.

Further, the guide track 36 comprises two guide rails 37. The guide track 36 is located at the side of the Geneva wheel 48. When the interaction member 8 moves from its first to its second position, the freely suspended tip 12 slides along the guide track 36 guided by the guide rails 37. The guide rails 36 allow for moving the freely suspended tip 12 along the side of the Geneva wheel 27 in a distal direction, in particular form the first position to the second position, without that the tip 12 interacts with the Geneva wheel 48. Instead, the freely suspended tip 12 is guided through the guide track 36 between the two guide rails 37 and thereby does not contact the Geneva wheel 48. The main part 31 of the interaction member 8 is moved in a plane that is offset to the guide rails 37 and, thereby, does not contact the guide rails 37.

When a dose delivery has been performed and the actuating element 54 and the interaction member 8 are in their respective second positions, the user may perform a second suction at the mouthpiece 6. However, in this case, no dose of the substance 2 is delivered as the metering chamber 40 is empty. The second suction also does not move the actuating element 54, as the actuating element 54 remains in its second position after a dose delivery until the cap 7 is engaged to the housing 3. Thereby, the interaction member 8 is not moved and, therefore, the state of the dose counting mechanism 5 is not altered by the second suction.

After dispensing the sub-quantity 14 of the medical substance 2, the interaction member 8 may be moved in the proximal direction when a user performs an after-dose delivery operation, e.g. the user may screw the cap 7 to the housing 3.

When the cap 7 is screwed on the housing 3, the actuating element 54 is moved into the proximal direction from its second position to its first position. As the interaction member 8 is coupled to the actuating element 54, the interaction member 8 is also moved in the proximal direction from its second position to its first position when the cap 7 is screwed onto the housing. Thereby, the freely suspended tip 12 of the interaction member 8 engages the Geneva wheel 48 and rotates the Geneva wheel 48 by 90°. This engagement is initiated by the entry guide lip 45 provided on the guide rail 37.

However, near the end of the proximal movement of the interaction member 8, the freely suspended tip 12 disengages from the receptacle 32 of the Geneva wheel 48 and slides into the guide track 36. The guide track 36 may comprise a guide feature, e.g. an exit guide lip 46 that disengages the tip 12 from the receptacle 32. The exit guide lip 46 may be flexible such that it is bendable allowing for a rotation of the Geneva wheel 48. At the same time, it is stiff enough to allow for the tip 12 to slide along the exit guide lip 46 out of the receptacle 32 of the Geneva wheel 27 and into the guide track 36.

Alternatively, the interaction member 8 may disengage from the receptacle 32 and engage into a proximal opening of the guide track 36 due to a resilient force. During its proximal movement the interaction member 8 slides along the surface of one of the guide rails 37, in particular the surface facing away from the guide track 36. Near the end of the proximal movement, the interaction member 8 is enabled to swing into the proximal opening of the guide track 36. Accordingly, the exit guide lip 46 is optional. Furthermore, also the entry guide lip 45 may be optional.

Accordingly, once the cap 7 is screwed on the housing 3, the interaction member 8 is moved in a proximal direction and the counting mechanism 5 is thereby updated. In case, the cap 7 is unscrewed from the housing 3, but no dose delivery is performed, the actuating element 54 remains in its first position and, thereby, the interaction member 8 remains in its first position. Accordingly, the interaction member 8 is not moved from its second position to its first position when the cap 7 is screwed back onto the housing 3. Accordingly, the Geneva wheel 48 is not rotated and the counted number of the counting mechanism 5 is not altered.

As can be seen in FIG. 3, the first, the second and the third counting members 28, 29, 30 comprise digits. Further, the housing 3 comprises a window 17. The window 17 is arranged such that one digit of each of the counting member 28, 29, 30 is visible. The digits of the counting members 28, 29, 30 visible through the window 17 form a counted number which is decremented by the counting mechanism 5. The counted number corresponds to the numbers of doses left in the device 1. The digit of the first counting member 28 visible in the window 17 may correspond to the units digit of the number of left doses, the digit of the second counting member 29 visible in the window 17 may correspond to the tens digit and the digit of the third counting member 30 visible in the window 17 may correspond to the hundreds digit.

In an alternate embodiment, the counted number visible in the window 17 may correspond to the number of doses that have been delivered by the device 1. Accordingly, in this embodiment, the counted number is incremented by the counting mechanism 5.

Moreover, the dose counting mechanism 5 may further comprise a balancing member not shown in FIGS. 2 and 3. The balancing member may be identical to the interaction member 8 regarding its shape, its material and its weight. In particular, the balancing member may be a deflectable stick. The balancing member may be coupled to the actuating element 54. Preferably, the balancing member is arranged at the actuating element 54 opposite to the interaction member 8. The balancing member ensures that the actuating element 54 is in a balanced position by providing a counterweight to the interaction member 8. In particular, the balancing member ensures that the actuating element 54 is not tilted towards the side of the interaction member. Further, the dose counting mechanism 5 may comprise a dummy Geneva wheel for interaction with the balancing member.

Furthermore, the inhalation device 1 comprises a lock-out mechanism 26. The lock-out mechanism 26 comprises a blocking element 39. The blocking element 39 may be a lock-out pin. The blocking element 39 is coupled to the housing 3 by a biasing member 41, e.g. by a spring. The blocking element 39 has a blocking position and an unblocking position.

In particular, FIG. 2 shows the lock-out mechanism 26 in a state wherein the blocking element 39 is in its unblocking position. The blocking element 39 is arranged between the housing 3 and the third counting member 30 of the dose counting mechanism 5 and does not interact with the interaction member 8.

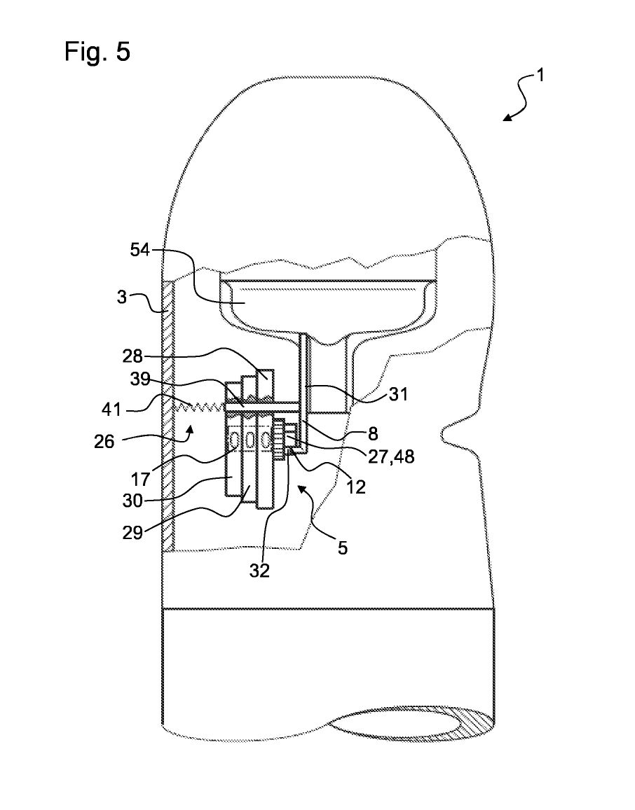

Each of the counting members 28, 29, 30 may have at least a first and a second state, in particular a first and a second orientation. Preferably, the counting members 28, 29, 30 are configured to prevent a movement of the blocking element 39 when the counting members 28, 29, 30 are in their respective first state (see FIG. 2). Further, when the counting members 28, 29, 30 are in their respective second state it may allow a movement of the blocking element 39 (see FIGS. 2 and 5).

As shown in FIG. 3, each of the counting members 28, 29, 30 comprises a path 42, 43, 44, e.g. formed by a hole through the counting member 28, 29, 30. The blocking element 39 is arranged such that it is enabled to move into the path 42, 43, 44 of the respective first, second or third counting member 28, 29, 30 when the paths 42, 43, 44 are aligned with the blocking element. The blocking member 39 may be enabled to move into the first, the second and the third coupling member successively. The blocking member 39 may be enabled to move to its blocking position by a movement comprising multiple sub-movements that may be separated in time. In particular, in a first sub-movement the blocking member 39 may move into the path 44 of the third counting member 30 and may be blocked from a further movement by the second counting member 29. In a second sub-movement the blocking member 39 may move into the path 43 of the second counting member 29 and may be blocked from a further movement by the first counting member 28. In a third sub-movement the blocking member 39 may move into the path 42 of the first counting member 28 and into engagement with the interaction member 8.

In particular, the blocking element 39 is arranged such that it is enabled to move into engagement with the path 44 of the third counting member 30 when the digit of the third counting member 30 visible through the window 17 equals zero. The blocking element 39 is movable into the path 44 of the third counting wheel 30 by a force exerted by the biasing member 41.

For example, the inhalation device 1 may comprise 150 doses of a medical substance 2. Accordingly, in its initial state the dose counting mechanism 5 shows the number “150” in the window 17. Once 51 doses have been delivered, the number “099” is shown in the window 17. Now the blocking element 39 is aligned with the path 44 of the third counting member 30 and is pushed by the biasing member 41 into the path 44.

However, when 51 doses have been delivered, the second counting member 29 does not show a zero in the window 17 in this state. Accordingly, the blocking element 39 is not aligned with the path 43 of the second counting member 29. Therefore, the blocking element 39 is prevented from moving into engagement with the path 43 of the second counting member 29. Accordingly, the blocking element 39 is still in an unblocking position as it dose not prevent an operation of the interaction member 8 and of the actuating element 54.

Moreover, when delivering further doses, the second counting member 29 is gradually rotated. When the second counting member 29 shows a zero in the window 17, the path 43 of the second counting member 29 is aligned with the blocking element 39. Accordingly, the blocking element 39 now is enabled to move into engagement with the path 43 of the second counting wheel 29. In particular, the blocking element 39 is pushed into the path 43 of the second counting member 29 by the biasing member 41.

When the last dose is delivered and the cap 7 has been engaged to the housing 3, the path 42 of the first counting member 28 is aligned with the blocking element 39. In this case, the digit of the first counting member 28 visible in the window 17 equals 0. Accordingly, “000” is visible in window 17. The blocking element 39 now engages the path 42 of the first counting member 28. The paths 42, 43, 44 of all counting members 28, 29, 30 are aligned to the blocking element 39.

Further, the blocking element 39 is now enabled to engage the interaction member 8. Thereby, the blocking element 39 prevents the interaction member 8 from being moved axially. Accordingly, a dose delivery operation is not possible anymore.

FIG. 5 shows the inhalation device 1 in a state when all available doses have been delivered. The counting members 28, 29, 30 have been rotated such that“000” is visible in the window 17 and the blocking element 39 is aligned with the paths of all counting members 28, 29, 30. Accordingly, the biasing member 41 has pushed the blocking element 39 into the path 42 of the first counting member 28. By the force exerted by the biasing member 41, the blocking element 39 is moved towards the interaction member 8 and engages with the interaction member 8 of the dose counting mechanism 5. The locking pin 39 is designed such that it prevents a further axial movement of the interaction member 8 once it is engaged with the interaction member 8. Accordingly, the blocking element 39 is in its blocking position.

In the blocking position, the blocking element 39 may be prevented from a movement relative to the housing of the device by its engagement with the first counting member 28. In particular, one end of the blocking element 39 may be engaged to the interaction member 8 and the other end of the blocking element 39 may be located in the path 42 and, thereby, may be engaged with the first counting member 28 such that the blocking element 39 is locked against a movement at least in distal direction. In particular, the first counting member 28 may be configured such that a rotation of the counting member 28 in a rotational direction opposite the rotational direction during updating the counted number is prevented. Thereby, the blocking element 39 locks the interaction member 8 against a movement at least in distal direction.

The blocking element 39 may engage with the interaction member 8 at the connection point of the main part 31 and the tip 12. In particular, the blocking element may engage with the interaction member 8 at a point slightly more distal than the tip 12.

In particular, the front face of the blocking element 39 facing towards the interaction member has an inwardly curved surface 47 which is adapted to the surface of the interaction member 8. Thereby, the blocking element 39 fixes the interaction member 8 such that it cannot be moved axially anymore. The interaction member 8 is locked in its first position, which is marked as “A” in FIG. 4. The interaction member 8 is prevented from a movement in a distal direction by the engagement of the blocking element 39.

Additionally or alternatively, the interaction member 8 may comprise a recess. The blocking element 39 may be enabled to engage into the recess and, thereby, to lock the interaction member 8 such that the interaction member 8 can not move axially anymore.

By axially fixing the interaction member 8, the blocking element 39 prevents a further movement of the actuating element 54. In particular, it is no longer possible to move the actuating element 54 axially in a distal direction. Thereby, a dose delivery of the inhalation device 1 is prevented.

When the actuating element 54 is moved from its first position to its second position, an audible and visible feedback is provided to the user. In particular, when the actuating element 54 is moved into its second position, the piston head 76 (see FIG. 1) may engage into a corresponding projection, thereby providing an audible feedback. Moreover, the housing 3 of the inhalation device 1 may comprise a small opening, allowing the user to see if the actuating element is in its first or its second position as the actuating element blocks the opening in its first position and, further, does not block the opening in its second position.

However, if a user performs a suction at the mouthpiece 6 after the blocking element 39 has engaged the interaction member 8, no audible or visible feedback is provided to the user as the actuating element 54 is not moved. Thereby, the user is informed that no dose has been delivered. Accordingly, the lock-out mechanism 26 alerts the user that the inhalation device 1 does not provide further doses of the medical substance 2.

Once the blocking element 39 locks the interaction member 8, a dose delivery of the inhalation device 1 is permanently prevented. It is not possible to unlock the blocking element 39 from the interaction member 8 unless the housing 3 of the device 1 is opened. When the blocking element 39 locks the interaction member 8, a user may dispose the inhalation device 1. Alternatively, a user or a trained person may refill new doses of a medical substance 2 or change a container comprising the medical substance 2 and thereafter unlock the lock-out mechanism 26.

Furthermore, the dose counting mechanism 5 may comprise a second spring which is not shown in FIGS. 2 to 5. The second spring is enabled to exert a force on the actuating element 54 in a distal direction. The actuating element 54 is moved from its first position to its second position if a user exerts a predetermined force by a suction airstream. However, the interaction member 8 interacting with the counter drive element and the balancing member interacting with the dummy counter drive element may increase the force required to move the actuating element 54 to its second position. The second spring allows adjusting the force required to move the actuating element 54 to its second position. In particular, the spring applies a force to the actuating element 54 in the direction of the mouthpiece 6, thereby decreasing the force necessary to move the actuating element 54 to its second position. Accordingly, the second spring compensates the effect of the interaction element 8 regarding the force required to move the actuating element 54.

The term “medical substance”, as used herein, may mean a pharmaceutical formulation containing at least one pharmaceutically active compound, for example for the treatment of obstructive airway or lung diseases such as asthma or chronic obstructive pulmonary disease (COPD), allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

1-15. (canceled)

16. Inhalation device for delivering at least one dose of a medicament, comprising a lock-out mechanism to prevent an operation of the inhalation device when a given number of doses has been delivered wherein a dose setting operation is still possible when the lock-out mechanism prevents the operation of the inhalation device.

17. Inhalation device according to claim 16, wherein the operation configured to be prevented by the lock-out mechanism is a dose delivery operation of the inhalation device.

18. Inhalation device according to claim 16, wherein the lock-out mechanism comprises a blocking element having a blocking position, wherein when the blocking element is in the blocking position the operation of the inhalation device is prevented.

19. Inhalation device according to claim 18, wherein the blocking element has an unblocking position allowing the operation of the inhalation device before the given number of doses has been delivered.

20. Inhalation device according to claim 19, configured such that the blocking element changes from its unblocking position to its blocking position when the given number of doses has been delivered.

21. Inhalation device according to claim 16, further comprising a dose counting mechanism enabled to count a number of doses.

22. Inhalation device according to claim 21, wherein the lock-out mechanism is enabled to prevent an operation of the inhalation device when the number counted by the dose counting mechanism has a first predefined numerical value.

23. Inhalation device according to claim 21, wherein the dose counting mechanism comprises a counting member providing a path.

24. Inhalation device according to claim 23, wherein the blocking element (39) is enabled to move to its blocking position when the path and the blocking element are aligned.

25. Inhalation device according to claim 23, wherein the dose counting mechanism comprises a second counting member providing a further path, and wherein the blocking element is enabled to move to its blocking position only when the paths of all counting members and the blocking element are aligned.

26. Inhalation device according to claim 21, wherein the dose counting mechanism comprises an interaction member and a coupling element, and wherein the interaction member is enabled to interact with the coupling element when the interaction member is moved in a first direction.

27. Inhalation device according to claim 26, wherein the dose counting mechanism further comprises a guide track preventing an interaction of the interaction member and the coupling element when the interaction member is moved in a second direction.

28. Inhalation device according to claim 16, comprising an actuating element being displacable from a first position to a second position, wherein the lock-out mechanism is enabled to prevent a displacement of the actuating element from its first position to its second position when the given number of doses has been delivered.

29. Inhalation device according to claim 18, wherein the lock-out mechanism further comprises a biasing member coupled to the blocking element, and wherein the biasing member exerts a biasing force on the blocking element.

30. Inhalation device according to claim 29, wherein the biasing member is configured to move the blocking element from its unblocking position to its blocking position when the given number of doses has been delivered.
